(19) United States
(12) Patent Application Publication
(10) Pub. No.: US 2005/0069397 A1
(43) Pub. Date: Mar. 31, 2005

(54) Locking mechanism for intramedullary nails

(76) Inventors: Ronen Shavit, Tel-Aviv (IL); Oren Globerman, Kfar-Shmaryahu (IL); Mordechay Beyar, Caesarea (IL)

Correspondence Address: William H Dippert;Reed Smith, 29th Floor, 599 Lexington Avenue, New York, NY, 10022-7650, US

(21) Appl. No.: 10/502,563

(22) PCT Filed: Jan. 22, 2003
(86) PCT No.: PCT/IL03/00052

(30) Foreign Application Priority Data
Jan. 23, 2002 (IL) 14783

(51) Int. Cl. F16B 15/00

(52) U.S. Cl. 411/457

(57) ABSTRACT

Intramedullary nail apparatus comprising: an intramedullary nail having an axis, a proximal end, a distal end, a first hole going through the nail at an angle to the axis, and a second hole going through said nail at the same or a different angle to the axis, with the second hole closer to the proximal end than the first hole; a first screw which goes through the first hole; a second screw which goes through the second hole; and a locking mechanism, accessible from the proximal end of the nail, operative to selectively lock in place the first screw.

### FIELD OF THE INVENTION

The present invention relates to the field of surgical nails used to repair bone fractures, in particular intramedullary nails used for fractures of the proximal femur.

### BACKGROUND OF THE INVENTION

Intramedullary nails, which are designed to repair fractures of the proximal femur, for example reconstructive nails or gamma nails, include a nail which is inserted into the medullar channel, and at least one lag screw, going through the nail at an angle, and through the neck of the femur, which is supposed to hold the neck of the femur and trochanter in place. In some designs there are two screws: a larger screw, and a smaller screw which goes through the nail closer to the proximal end than the larger screw. Depending on the type of fracture, it may be desirable to limit the motion of the screw or screws relative to the nail. The lack of a mechanism to do this limits the usefulness of these nails. For example, it was reported by Florin et al [N. Florin, T. Roessler and K. Westermann, “Possible complications of internal fixation using the Gamma Nail,” in The Gamma Locking Nail: Ten Years Results and Surgical Experience, R. H. Gahr, K.-S. Leung, M. P. Rosenwasser and W. Roth, eds., Einhorn-Presse Verlag, Germany, p. 244] that the screws sometimes migrate after surgery, damaging the hip joint or allowing the trochanter to collapse.

In existing nails which use a single lag screw, there may be a small set screw, set into the proximal end of the nail by the surgeon, which can be tightened to keep the lag screw from moving, or to limit its motion, once the nail is in place. (Often it is not desired to hold the lag screw rigidly in place, but only to limit the range of its motion with respect to the nail, since it is believed that the bone will heal better if it is allowed to move and bear some of the load. “Locking” the screw as used here does not necessarily mean preventing it from moving at all, but it may simply mean limiting its motion.) In nails which use two lag screws, it is not possible to lock both screws, since the screw that is closer to the proximal end of the nail blocks access to the screw that is further from the proximal end of the nail. Patent WO 01/39679, for example, describes a nail with two screws, a smaller screw closer to the proximal end of the nail and a bigger screw further from the proximal end, in which there is no mechanism to lock the screws.

PCT Publication WO 01/54598 describes an intramedullary nail, the distal portion of which is hollow. Once the nail is in place, fluid is forced into the nail under pressure, causing the distal portion to expand radially and lock against the medullar channel. Once the bone is healed, the fluid can be drawn out, causing the nail to shrink back radially, so that it can be easily removed from the medullar channel. This allows marrow to grow back.

### SUMMARY OF INVENTION

An aspect of some embodiments of the invention relates to a nail with two screws going through it, one screw closer to the proximal end of the nail than the other screw, and a locking mechanism, accessible from the proximal end, which locks the screw that is further from the proximal end. Optionally, the locking mechanism does not interfere with the screw that is closer to the proximal end. Alternatively, the same locking mechanism, or another locking mechanism also accessible from the proximal end of the nail, also locks the screw closer to the proximal end. In some embodiments it is possible to lock either screw, or both of them, or neither of them, according to the judgment of the surgeon.

An aspect of some embodiments of the invention relates to a nail with at least one screw going through it, and a locking mechanism for at least one screw, which locking mechanism is an integral part of the nail, and cannot be removed from the nail by mistake.

An aspect of some embodiments of the invention relates to a nail with at least one screw going through it, and a channel in the nail for injecting liquid into or through the nail, in which the channel circumvents the at least one screw. Optionally, the distal portion of the nail is hollow, and the channel is used to inject liquid under pressure into the distal portion of the nail, causing it to expand radially and lock the nail against the medullar channel. Alternatively or additionally, the channel is used to inject some kind of cement to hold one or more of the one or more screws in place or to hold the nail in place.

An aspect of some embodiments of the invention relates to a nail with at least one screw, a locking mechanism for locking one or more of the at least one screws, a channel for injecting a liquid into or through the nail, and a valve for closing off the channel, where the channel is an integral part of the locking mechanism. Optionally, engaging the locking mechanism to lock a screw also closes the valve. Alternatively, engaging the locking mechanism is independent of closing the valve. Optionally, the distal portion of the nail is hollow, and the channel is used to inject liquid under pressure into the distal portion of the nail, causing it to expand radially and lock the nail against the medullar channel. In this case, closing the valve is necessary in order to keep the liquid under pressure. Alternatively or additionally, the channel is used to inject some kind of cement to hold one or more of the one or more screws in place, or to hold the locking mechanism in place.

In some embodiments of the invention, one or both of the screws are hollow and liquid is injected into them under pressure, to cause them to expand and to anchor them in the bone, in addition to or instead of using screw threads to anchor them in the bone. Even when they are lacking screw threads, these devices are still referred to herein as “screws” because they play the same role as the screws in other intramedullary nails. In these embodiments of the invention, the larger screw is also sometimes referred to as a “hip peg” and the smaller screw is also sometimes referred to as a “hip pin.”

There is thus provided, in accordance with an embodiment of the inventions, an intramedullary nail apparatus comprising:

an intramedullary nail having an axis, a proximal end, a distal end, a first hole going through the nail at an angle to the axis, and a second hole going through said nail at the same or a different angle to the axis, with the second hole closer to the proximal end than the first hole;
a first screw which goes through the first hole;
a second screw which goes through the second hole; and
a locking mechanism, accessible from the proximal end of the nail, operative to selectively lock in place the first screw.

In an embodiment of the invention, the locking mechanism operates to lock into place both the first screw and the second screw.

Optionally, locking the first screw causes the second screw to be locked at the same time.

Alternatively, the locking mechanism selectively locks one of the screws without locking the other screw.

Alternatively, there is a second-screw locking mechanism, accessible from the proximal end of the nail, operative to lock in place the second screw, which second-screw locking mechanism operates independently of the locking mechanism.

In an embodiment of the invention, at least part of the locking mechanism is located in the interior of the nail between the proximal end and the first hole.

Optionally, the locking mechanism comprises a linear adapter which moves axially when it is rotated, and a stem, adjacent to the linear adapter and closer to the distal end, which moves axially without rotating, when the linear adapter moves axially.

Optionally, the linear adapter is threaded and screws into an opening at the proximal end of the nail.

Optionally, the stem defines a hole which substantially or completely overlaps the second hole of the nail, but is longer than the second hole in the direction of the axis of the nail.

Optionally, the stem comprises a tab which extends into the first hole enough to reach the first screw when the locking mechanism operates to lock the first screw in place.

Optionally, the first screw includes a slot, and the tab of the stem fits into the slot of the first screw when the locking mechanism operates to lock the first screw in place.

Alternatively, the second screw defines at least one by-pass hole, and the locking mechanism extends through one of the at least one by-pass holes when the locking mechanism operates to lock in place the first screw.

Alternatively, the second screw defines a slit, and the locking mechanism extends through the slit when the locking mechanism operates to lock in place the first screw.

Alternatively, the locking mechanism passes to one side of the second screw, when the locking mechanism operates to lock in place the first screw.

Optionally, there is a third screw which passes through the nail at an angle to the axis and closer to the proximal end of the nail than the first hole, and the locking mechanism passes between the second screw and the third screw when the locking mechanism operates to lock in place the first screw.

In an embodiment of the invention, there is a channel entrance open to the exterior of the nail, at least one channel exit closer to the distal end of the nail than the channel entrance is, and a channel adapted for passage of fluid from the channel entrance to the one or more channel exits.

Optionally, the channel is linked to inject cement to bond in place the second screw.

Optionally, the channel is comprised in the locking mechanism.

Optionally, the channel circumvents the first hole.

There is further provided, in accordance with an embodiment of the invention, an intramedullary nail apparatus comprising:
an intramedullary nail with an axis, a proximal end, a distal end, and a first hole going through said nail at an angle to the axis;
a first screw which goes through the first hole; and
a locking mechanism, operative to lock in place the first screw, and which mechanism includes a channel entrance open to the exterior of the nail, at least one channel exit closer to the distal end of the nail than the channel entrance is, and a channel adapted for passage of fluid from the channel entrance to the one or more channel exits.

In an embodiment of the invention, the intramedullary nail includes a hollow region near the distal end, capable of anchoring the nail in place when fluid is injected into said hollow region, and the locking mechanism comprises a valve which operates to seal the channel.

Optionally, the locking the locking mechanism closes the valve.

Alternatively, the locking mechanism operates independently of closing the valve.

Optionally, the channel is linked to inject fluid into the hollow region.

Alternatively or additionally, the channel is linked to inject cement to bond in place one or both of the nail and the first screw.

There is further provided, in accordance with an embodiment of the invention, an intramedullary nail apparatus comprising:
an intramedullary nail with an axis, a proximal end, a distal end, and a first hole going through said nail at an angle to the axis;
a first screw which goes through the first hole;
a channel entrance open to the exterior of the nail, and closer to the proximal end of the nail than the first hole is;
one or more channel exits closer to the distal end of the nail than the first hole is; and
a channel adapted for passage of fluid, which channel runs inside the nail from the channel entrance to the one or more channel exits, circumventing the first hole.

In an embodiment of the invention, the intramedullary nail includes a hollow region near the distal end, which hollow region expands and anchors the nail in place when fluid is injected into said hollow region.

Optionally, the channel is linked to inject fluid into the hollow region.

Additionally or alternatively, the channel is linked to inject cement to bond in place one or both of the nail and the first screw.

In an embodiment of the invention, there is a motion limiting element to prevent the locking mechanism from separating from the nail.

There is further provided, in accordance with an embodiment of the invention, an intramedullary nail apparatus comprising:
an intramedullary nail with an axis, a proximal end, a distal end, and a first hole going through said nail at an angle to the axis;
a first screw which goes through the first hole;
a locking mechanism, operative to lock in place the first screw; and
a motion limiting element that prevents the locking mechanism from separating from the nail.

Optionally, at least part of the locking mechanism is inside the nail, and the motion limiting element comprises:
a nail-stopping screw;
a nail-stopping screw hole in the side of the nail going from the outside of the nail to the inside of the nail; and
a nail-stopping indentation in the side of the locking mechanism; and the nail-stopping screw goes through the nail-stopping screw hole and engages the nail-stopping indentation when the motion limiting element operates to prevent the locking mechanism from separating from the nail.

Optionally, the nail-stopping indentation is longer in the direction of the nail axis than the width of the portion of the nail-stopping screw than engages in said slot, and the motion limiting element has at least one mode of operation in which the locking mechanism is free to move axially by a given distance but is prevented from separating from the nail.

Optionally, the nail-stopping indentation comprises a slot of limited width in the azimuthal direction.

Alternatively, the nail-stopping indentation extends around the entire circumference of the locking mechanism.

Optionally, locking the first screw comprises not allowing the first screw to move in the direction of its axis.

Alternatively, locking the first screw comprises allowing the first screw to move only a limited distance in the direction of its axis.

There is further provided, in accordance with an embodiment of the invention, a method for installing an intramedullary nail in the proximal femur, comprising:
inserting the nail, distal end first, into the medullar channel of the femur from the head of the femur;
inserting a first screw through a first hole in the nail, through the head of the femur and through the trochanter, at an angle to the axis of the nail;
inserting a second screw through a second hole in the nail, closer to the proximal end of the nail than the first screw; and
locking the first screw in place using a locking mechanism accessible from the proximal end of the nail.

### BRIEF DESCRIPTION OF THE DRAWINGS

Exemplary embodiments of the invention are described in the following sections with reference to the drawings. The same reference numbers are used for the same or similar features on different drawings. Some of the drawings may not be drawn to scale.

FIG. 1A is a perspective view, and FIG. 1B is a side cross-sectional view, showing a nail with holes for inserting two screws, and showing one of the screws inserted into the nail, according to an exemplary embodiment of the invention;

FIG. 2 is a perspective view according to the same embodiment as FIGS. 1A and 1B, showing the disassembled parts of the nail itself, without the screws;

FIGS. 3A and 3B show side cross-sectional views of the nail seen from two different directions, according to the same embodiment as FIGS. 1A and 1B, showing the details of the locking mechanism, valve and channels;

FIG. 4 is a schematic side view of the nail, showing locking mechanisms for two screws, according to a different embodiment of the invention;

FIGS. 5A, 5B, and 5C are perspective views of the nail according to three different embodiments of the invention; and

FIGS. 5D and 5E are perspective views of the locking mechanism and the second screw (or hip pin), showing mechanisms by which the locking mechanism locks the second screw, according to two different embodiments of the invention.

### DETAILED DESCRIPTION OF EXEMPLARY EMBODIMENTS

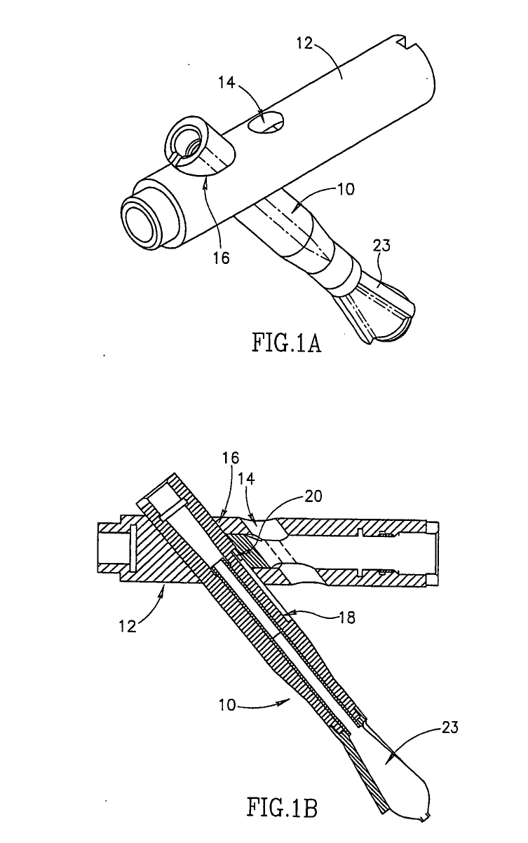

FIGS. 1, 2, and 3 illustrate, according to an embodiment of the invention, a nail 12 with holes 14 and 16 for inserting two screws, with a locking mechanism 22, accessible from the proximal end of the nail, which locks in place the larger screw 10, which is further away from the proximal end, without interfering with the smaller screw, which is closer to the proximal end. The larger screw is also called a hip peg and the smaller screw is also called a hip pin. In other embodiments of the invention, the two screws are the same size, or the larger screw is closer to the proximal end.

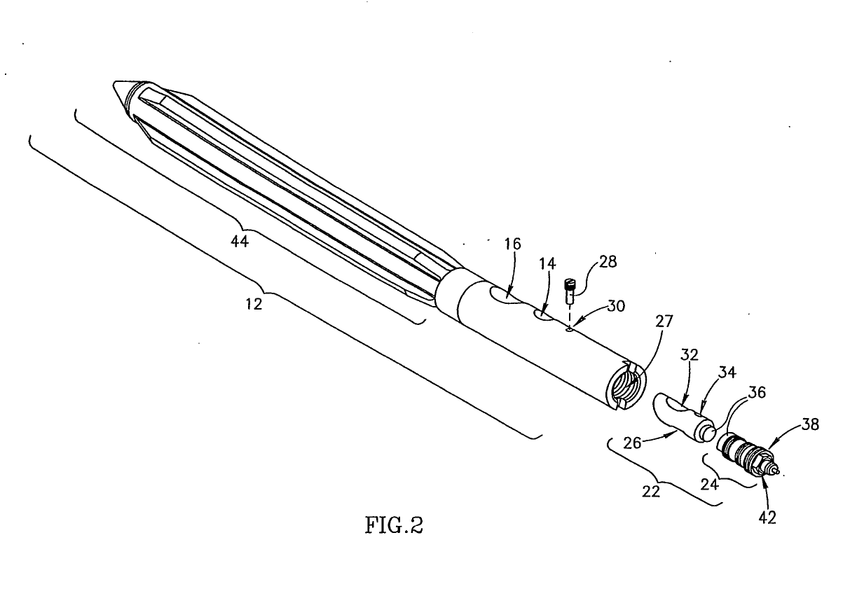

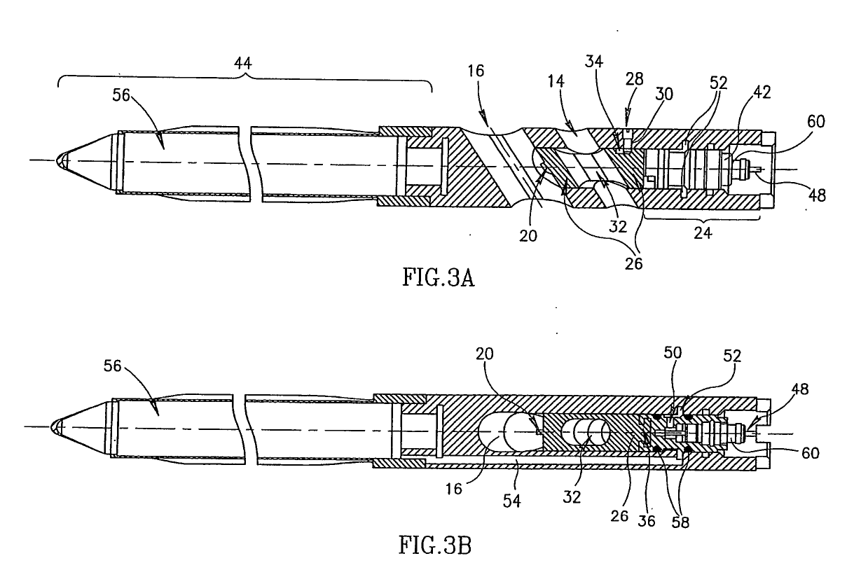

FIG. 1A is an external view of the proximal portion of nail 12, with smaller hole 14, closer to the proximal end, and larger hole 16 further from the proximal end. Hip peg 10 is shown inserted into hole 16. No hip pin is shown in hole 14, for clarity. A cross-sectional side view of the same embodiment is shown in FIG. 1B, in order to show how the hip peg is locked. The hip peg has a slot 18, and there is a tab 20, at the end of locking mechanism 22, which fits into slot 18, preventing hip peg 10 from coming out of hole 16. (The rest of locking mechanism 22 is not shown in FIG. 1B, but is shown in FIGS. 2 and 3.) If slot 18 is 20 mm longer, for example, than tab 20, in a direction along the axis of the hip peg, then the hip peg is able to move back and forth along its axis a distance of 20 mm, even when it is locked. Optionally, slot 18 is also wider azimuthally than tab 20, so that the hip peg is free to rotate by a limited angle when tab 20 is inserted into slot 18, in addition to being free to move a limited distance along its axis. Alternatively or additionally, slot 18 is spiral-shaped, so that the hip peg is free to move along its axis by a limited amount, only if it also rotates at the same time. Optionally, there is a second slot in hip peg 10, shorter than slot 18, so that hip peg 10 would not be free to move at all when tab 20 is inserted into the second slot. Then hip peg 10 can either be locked completely, or partially locked with freedom to move a limited amount. Optionally, the second slot is inside slot 18. Alternatively, the second slot is separate from slot 18. Optionally, the inside of slot 18, or another slot, is lined with a resilient material. Then, when tab 20 is inserted into that slot, hip peg 10 can move a limited amount, but there is a spring force resisting its motion. Such an arrangement has the advantage that the stress is not entirely born by the nail, or entirely born by the bone, but is distributed between the nail and the bone, which may promote optimal healing of the bone. Alternatively or additionally, such a spring force can be produced by making the nail or locking mechanism out of a compliant material.

In the embodiment of the invention shown in FIGS. 1A and 1B, hip peg 10 has a hollow region 23 at its end, which expands radially, anchoring it in place in the bone, when it is filled with liquid under pressure. Alternatively or additionally, screw threads or any other anchoring mechanism is used to anchor hip peg 10 in the bone.

FIG. 2 shows nail 12 with the disassembled parts of locking mechanism 22, in order to illustrate how the locking mechanism is able to lock the hip peg without interfering with the hip pin. Locking mechanism 22 comprises a linear adapter 24 and a stem 26. There is an opening 27 at the proximal end of the nail. Adapter 24 has threads 38 which match threads inside opening 27. When adapter 24 is inserted into opening 27 and the threads are engaged, turning adapter 24 with a screw-driver makes adapter 24 move axially with respect to the nail. Adapter 24 connects to stem 26, using coupling mechanism 36. The coupling mechanism allows adapter 24 to rotate freely with respect to stem 26 while it is coupled. In particular, the coupling mechanism allows adapter 24 to push or pull stem 26 axially, without requiring stem 26 to rotate, when adapter 24 is turned by a tool such as a screw-driver and moves axially with respect to the nail. As will be explained, the lack of rotation of stem 26 makes it possible for the locking mechanism not to interfere with the hip pin. In this embodiment, coupling mechanism 36 does not allow adapter 24 to become disconnected from stem 26 as long as they remain inside the nail, since the narrowness of the opening in the nail keeps adapter 24 and stem 26 from moving very far transversely to the axis of the nail. Adapter 24 and stem 26 are coupled together before they are inserted into the nail. Adapter 24 optionally has a hex nut 42 at its end, which allows adapter 24 to be turned by a socket screwdriver inserted into opening 27. Alternatively, another mechanism known to the art is used to turn adapter 24 while it is inside the nail.

Locking mechanism 22 is designed to avoid interfering with the hip pin. This is accomplished by means of a hole 32 in stem 26. Hole 32 is longer than hole 14 in the direction of the axis of the nail. When locking mechanism 22 is operating to lock hip peg 10, hole 32 overlaps hole 14, and it is possible to insert the hip pin through hole 14 and hole 32. The fact that hole 32 is longer than hole 14 allows the two holes to remain overlapping axially when stem 26 moves a limited distance axially. Optionally, hole 32 is long enough so that it does not interfere with the hip pin, when stem 26 moves axially from a position where hip peg 10 is completely unlocked, to a position where hip peg 10 is fully locked. This may be seen in FIG. 3A, which shows a cross-sectional view of locking mechanism 22 assembled inside the nail.

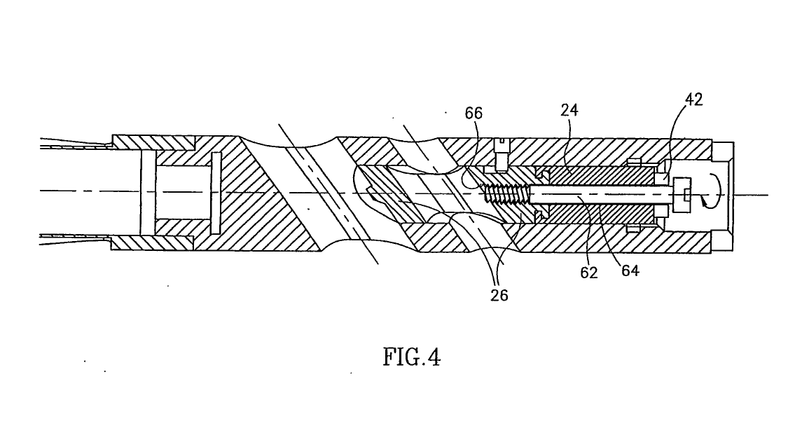

Alternatively, hole 32 has a tab inside it, similar to tab 20, which engages in a slot in the hip pin, similar to slot 18 in the hip peg, and locks the hip pin in place when the hip peg is locked in place. The optional features mentioned previously for slot 18 are also options for the slot in the hip pin. Also, optionally, the distance between tab 20 and the tab inside hole 32 is such that the hip peg and hip pin do not both lock at the same time, but one of them locks first, as adapter 24 is screwed down, and then the other one locks as adapter 24 is screwed down further. Alternatively, if it is not important to allow the hip pin freedom to move a limited distance when it is locked, there is no tab inside hole 32, and the inside of hole 32 hits the side of the hip pin and locks it in place, or there is a tab or some other projection inside hole 32 which hits the side of the hip pin and locks it in place. In still another alternative, shown in FIG. 4, the hip pin and hip peg each has its own locking mechanism, and the hip pin and hip peg may each be locked or not, regardless of whether the other one is locked.

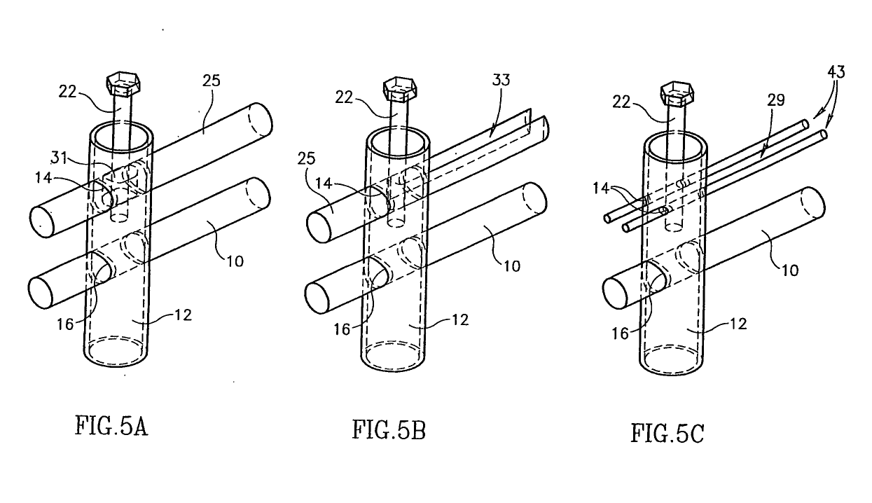

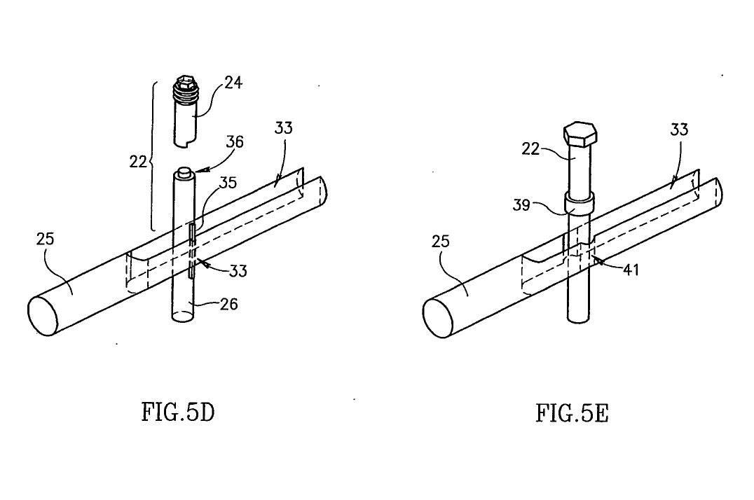

In the embodiments of the invention shown in FIGS. 1A through 4, locking mechanism 22 locks hip peg 10 while avoiding interfering with the hip pin, because hole 32 in locking mechanism 22 allows locking mechanism 22 to go around the hip pin without touching it. Alternatively or additionally, as shown in FIG. 5A, there is a hole 31 in a hip pin 25, which allows hip pin 25 to go around locking mechanism 22 without interfering with locking mechanism 22. If it is desired for hip pin 25 to have a limited range of axial motion with respect to nail 12, rather than being rigidly held in place, then hole 31 is made wider than locking mechanism 22, by an amount equal to the desired range of motion. Optionally, hole 31 is elongated, so that hip pin 25 is movable over a range of axial positions. Alternatively or additionally, there are two or more holes 31 at different locations along hip pin 25, so that hip pin 25 can be locked at two or more discrete positions. Optionally, there are markings on the outside of hip pin 25 that also reach outside the bone, which show the surgeon how far to insert hip pin 25 into hole 14, and/or how far to turn it, so that hole 31, or one of several holes 31, is positioned and oriented in such a way that locking mechanism 22 goes through it. Alternatively, if hip pin 25 does not need to be rotated, then hip pin 25, or at least the proximal end of hip pin 25, optionally has a non-circular cross-sectional shape, and hole 14 has the same non-circular shape, so that hip pin 25 can only be inserted into hole 14 with the correct orientation which allows locking mechanism 22 to go into hole 31. Even if hip pin 25 does need to be rotated, a non-circular cross-sectional shape of hip pin 25 optionally shows the surgeon the orientation that hip pin 25 needs to have when it is finished rotating, in order to insert locking mechanism 22. If there is only one hole 31 in hip pin 25, the proximal end of hip pin 25 is optionally made wider than hole 14, or there is a protusion on the side of hip pin 25, so that hip pin 25 can only be inserted far enough into hole 14 to align hole 31 with locking mechanism 22.

In still another alternative, shown in FIG. 5B, there is a slit 33 in hip pin 25 instead of a hole, extending from the distal end of hip pin 25 up past the part of hip pin 25 that crosses the path of nail 12 when hip pin 25 is in place. Having such a slit instead of a hole in hip pin 25 facilitates the insertion of hip pin 25 into nail 12 after the end of locking mechanism 22 is pushed past hole 14. Having a slit instead of a hole in hip pin 25 also means that hip pin 25 is adjustable to any desired axial position over a broad range, and that hip pin 25 need not be locked when hip peg 10 is locked by locking mechanism 22. It is still possible if desired to use locking mechanism 22 to lock hip pin 25, for example by a tab attached to the side of locking mechanism 22 which fits into a slot, or one of a series of slots, to the side of slit 33 in hip pin 25.

In the examples of FIGS. 5A and 5B, hip pin 25 cannot be rotated if it is one solid piece (for example, to screw it down if it uses screw threads) once locking mechanism 22 for hip peg 10 is pushed past hip pin 25. Alternatively, as shown in FIG. 5C, there are two parallel hip pins 43, with a space 29 in between them, and locking mechanism 22 goes through space 29 between the two hip pins, not interfering with the hip pins. This allows each of the hip pins to rotate even after locking mechanism 22 is pushed past them. Alternatively, there is only one hip pin 25, without a hole or a slit, and locking mechanism 22 for hip peg 10 also does not have a hole, but one or both of locking mechanism 22 and hip pin 25 do not intersect the axis of nail 12, but pass to the side of the axis, so locking mechanism 22 and the hip pin 25 do not interfere with each other. FIG. 5C would illustrate such a configuration if one of the two hip pins 25 in FIG. 5C, and the corresponding hole of the two holes 14, were removed from the drawing.

It is possible, if desired, to use locking mechanism 22 to lock hip pin 25, even if there is a very elongated hole 31 in an embodiment similar to that shown in FIG. 5A, so that the axial motion of hip pin 25 is not limited very much, and even in embodiments similar to those shown in FIG. 5B and FIG. 5C where the axial motion of hip pin 25 is not limited at all, or is limited only in one direction. Two exemplary ways to do this are shown in FIGS. 5D and 5E. FIGS. 5D and 5E illustrate such mechanisms for the case of a hip pin with a slit 33 in it, as in FIG. 5B, but similar mechanisms could be used for the cases of a hip pin with an elongated hole 31 in it, as in FIG. 5A, and for the case of two hip pins, as in FIG. 5C. In FIG. 5D, locking mechanism 22 comprises an adapter 24, a coupling mechanism 36, and a stem 26, similar to the locking mechanism in FIG. 2. This allows adapter 24 to be threaded and to rotate, pushing stem 26 axially, without stem 26 rotating. Stem 26 optionally has a tab 35 which fits into a slot 37 on the side of slit 33 in hip pin 25, locking hip pin 25 in place when tab 35 is pushed into slot 37. Optionally there is more than one slot 37 on the side of slit 33 so that hip pin 25 is lockable at more than one axial position. An alternative example of a mechanism to lock hip pin 25 is shown in FIG. 5E. Locking mechanism 22 is one solid piece, with a protrusion 39 which extends azimuthally around locking mechanism 22. Indentation 41 in the side of slit 33 matches the shape of protrusion 39. When protrusion 39 is pushed into indentation 41, hip pin 25 is locked axially. Because protrusion 39 is cylindrically symmetric around locking mechanism 22, locking mechanism 22 can rotate even while protrusion 39 is locking hip pin 25. Optionally, there is more than one indentation 41 in hip pin 25, so that hip pin 25 can be locked at more than one axial position. Optionally, tab 35 engages in slot 37 to lock hip pin 25 in FIG. 5D, and protrusion 39 engages indentation 41 to lock hip pin 25 in FIG. 5E, at the same time as locking mechanism 22 locks hip peg 10. Alternatively, hip pin 25, is locked before hip peg 10 is locked, as locking mechanism 22 is pushed down into nail 12, or hip peg 10 is locked before hip pin 25 is locked, so that it is possible for the surgeon to lock either one or both of hip pin 25 and hip peg 10.

In an exemplary embodiment of the invention shown in FIGS. 2 and 3, there is a device to keep adapter 24 from being unscrewed so far that locking mechanism 22 becomes separated from the nail. There is a nail-stopping screw 28, which goes through a hole 30 in the nail, and engages in a slot 34 in stem 26. Optionally a peg or a projection of some kind is used instead of a screw. After locking mechanism 22 is assembled and inserted into the nail, nail-stopping screw 28 is inserted into hole 30. Slot 34 is long enough axially so that, when nail-stopping screw 28 engages slot 34, stem 26 is free to move axially from a position where tab 20 is not engaged at all in slot 18 (but adapter 24 is still screwed part of the way into opening 27) to the position where tab 20 is completely engaged in slot 18. Nail-stopping screw 28 optionally serves another function: it prevents stem 26 from accidentally turning and becoming misaligned, before the hip pin has been inserted into hole 14. Locking mechanism 22 is assembled and inserted into nail 12 before the nail is inserted into the medullar channel of the femur during surgery, and before the hip peg and hip pin are inserted into the nail. Making the locking mechanism an integral part of the nail in this way means that there is less chance of the locking mechanism getting lost during surgery. Also, it is not necessary for the surgeon to repeatedly probe the injured area trying to insert the locking mechanism into the nail after the nail is in place in the bone, saving time and possibly avoiding additional trauma.

FIGS. 2 and 3 also illustrate a mechanism for anchoring the nail in place in the medullar channel of the femur. Nail 12 has a hollow distal portion 44. Once the nail is in place in the medullar channel, water or another fluid is injected under pressure into distal portion 44. The fluid pressure causes distal portion 44 to expand radially, locking it into place in the medullar channel, as described in PCT Publication WO 01/54598.

In an exemplary embodiment of the invention shown in FIGS. 3A and 3B, fluid is injected through the adapter and/or through a channel that bypasses holes 14 and 16. FIGS. 3A and 3B are side cross-sectional views of nail 12 with adapter 24 and stem 26 in place. The fluid is injected into a channel 48 at the proximal end of adapter 24. From there the fluid flows out an opening 50 (visible only in FIG. 3B) in the side of adapter 24, into a channel 52 going around the inside of nail 12 adjacent to opening 50, and down a tube 54 (visible only in FIG. 3B), which runs inside the wall of nail 12, skirting hole 14 and hole 16, and enters a hollow region 56 inside distal portion 44 of nail 12. Optionally, there are two o-rings 58, between valve-adapter 24 and the inner wall of nail 12, which keep the fluid from leaking out of channel 52, in either the proximal or distal direction.

Once the fluid is injected, a valve 60, at the end of adapter 24 adjacent to the opening of channel 48, is closed off by turning it, to keep the fluid sealed in and under pressure. Optionally, the action of screwing in adapter 24 to operate locking mechanism 22 also closes valve 60. Alternatively, valve 60 is closed independently of locking mechanism 22, using a different tool than the tool used to screw in adapter 24, or using the same tool in a different way.

Alternatively or additionally, the channels are used to inject cement through the nail, in order to hold the hip peg or hip pin in place, or to hold the nail in place in the medullar channel.

FIG. 4 is a schematic diagram showing a different embodiment of the invention, in which there is a locking mechanism for the hip pin, which acts independently of locking mechanism 22. In FIG. 4, locking mechanism 22 consists of a linear adapter 24, playing the same role as adapter 24 in FIGS. 2 and 3, and a stem 26. The locking mechanism for the hip pin comprises a lag screw 62 which is inserted into a hole 64 going along the axis of locking mechanism 22. Hole 64 extends all the way through adapter 24, and up to hole 32 in stem 26. Threads 66 in the part of hole 64 going through stem 26 match the threads of lag screw 62. When the lag screw is screwed down all the way, it extends a short distance into hole 32, and hits the hip pin. Optionally, one tool, for example a regular screw driver, is used to screw down lag screw 62 and lock the hip pin, while a different tool for example a socket screw driver fitting around hex nut 42, locks the hip peg. Thus, it is possible to lock either the hip peg or the hip pin or both of them or neither of them. Optionally there is a slot in the hip pin which the end of lag screw 62 fits into. This slot in the hip pin plays an analogous role to slot 18 in the hip peg, allowing the hip pin to move a limited distance when it is locked. The design options described previously for slot 18 in the hip peg are optionally usable for the slot in the hip pin.

In the embodiment shown in FIG. 4 there is no valve and there are no channels for injecting liquid into the distal portion of the nail, but optionally such parts exist, in addition to a locking mechanism for the hip pin. For example, the channel for fluid and the valve could be located inside lag screw 62. Alternatively, they are located in adapter 24 to the side of lag screw 62, or the channel goes through both lag screw 62 and adapter 24.

As used herein, the terms “have”, “include” and “comprise” or their conjugates mean “including but not limited to.” The term “lock” or “lock in place” when used regarding a hip peg, hip pin or screw, does not mean that the hip peg, hip pin or screw cannot move at all, but only means that its range of motion is limited. The term “screw” is sometimes used to mean any device that goes through an intramedullary nail at an angle to the axis of the nail to help stabilize a fracture, and could include a hip peg or hip pin, even if it is not threaded.

1. Intramedullary nail apparatus comprising:
an intramedullary nail having an axis, a proximal end, a distal end, a first hole going through the nail at an angle to the axis, and a second hole going through said nail at the same or a different angle to the axis, with the second hole closer to the proximal end than the first hole;
a first screw which goes through the first hole;
a second screw which goes through the second hole; and
a locking mechanism, accessible from the proximal end of the nail, operative to selectively lock in place the first screw.

2. Apparatus according to claim 1 wherein the locking mechanism operates to lock into place both the first screw and the second screw.

3. Apparatus according to claim 2 wherein locking the first screw causes the second screw to be locked at the same time.

4. Apparatus according to claim 2 wherein the locking mechanism selectively locks one of the screws without locking the other screw.

5. Apparatus according to claim 1, and including a second-screw locking mechanism, accessible from the proximal end of the nail, operative to lock in place the second screw, which second-screw locking mechanism operates independently of the locking mechanism.

6. Apparatus according to claim 1, wherein at least part of the locking mechanism is located in the interior of the nail between the proximal end and the first hole.

7. Apparatus according to claim 6, wherein the locking mechanism comprises a linear adapter which moves axially when it is rotated, and a stem, adjacent to the linear adapter and closer to the distal end, which moves axially without rotating, when the linear adapter moves axially.

8. Apparatus according to claim 7 wherein the linear adapter is threaded and screws into an opening at the proximal end of the nail.

9. Apparatus according to claim 7 wherein the stem defines a hole which substantially or completely overlaps the second hole of the nail, but is longer than the second hole in the direction of the axis of the nail.

10. Apparatus according to claim 7 wherein the stem comprises a tab which extends into the first hole enough to reach the first screw when the locking mechanism operates to lock the first screw in place.

11. Apparatus according to claim 10 wherein the first screw includes a slot, and the tab of the stem fits into the slot of the first screw when the locking mechanism operates to lock the first screw in place.

12. Apparatus according to claim 6 wherein the second screw defines at least one by-pass hole, and the locking mechanism extends through one of the at least one by-pass holes when the locking mechanism operates to lock in place the first screw.

13. Apparatus according to claim 6 wherein the second screw defines a slit, and the locking mechanism extends through the slit when the locking mechanism operates to lock in place the first screw.

14. Apparatus according to claim 6, wherein the locking mechanism passes to one side of the second screw, when the locking mechanism operates to lock in place the first screw.

15. Apparatus according to claim 14, and including a third screw which passes through the nail at an angle to the axis and closer to the proximal end of the nail than the first hole, wherein the locking mechanism passes between the second screw and the third screw when the locking mechanism operates to lock in place the first screw.

16. Apparatus according to claim 1, and including a channel entrance open to the exterior of the nail, at least one channel exit closer to the distal end of the nail than the channel entrance is, and a channel adapted for passage of fluid from the channel entrance to the one or more channel exits.

17. Apparatus according to claim 16 wherein the channel is linked to inject cement to bond in place the second screw.

18. Apparatus according to claim 16 wherein the channel is comprised in the locking mechanism.

19. Apparatus according to claim 16, wherein the channel circumvents the first hole.

20. Intramedullary nail apparatus comprising:
an intramedullary nail with an axis, a proximal end, a distal end, and a first hole going through said nail at an angle to the axis;
a first screw which goes through the first hole; and
a locking mechanism, operative to lock in place the first screw, and which mechanism includes a channel entrance open to the exterior of the nail, at least one channel exit closer to the distal end of the nail than the channel entrance is, and a channel adapted for passage of fluid from the channel entrance to the one or more channel exits.

21. Apparatus according to claim 20 wherein the intramedullary nail includes a hollow region near the distal end, capable of anchoring the nail in place when fluid is injected into said hollow region, and wherein the locking mechanism comprises a valve which operates to seal the channel.

22. Apparatus according to claim 21 wherein locking the locking mechanism closes the valve.

23. Apparatus according to claim 21 wherein the locking mechanism operates independently of closing the valve.

24. Apparatus according to claim 21 wherein the channel is linked to inject fluid into the hollow region.

25. Apparatus according to claim 20 wherein the channel is linked to inject cement to bond in place one or both of the nail and the first screw.

26. Intramedullary nail apparatus comprising:
an intramedullary nail with an axis, a proximal end, a distal end, and a first hole going through said nail at an angle to the axis;
a first screw which goes through the first hole;
a channel entrance open to the exterior of the nail, and closer to the proximal end of the nail than the first hole is;
one or more channel exits closer to the distal end of the nail than the first hole is; and
a channel adapted for passage of fluid, which channel runs inside the nail from the channel entrance to the one or more channel exits, circumventing the first hole.

27. Apparatus according to claim 26 wherein the intramedullary nail includes a hollow region near the distal end, which hollow region expands and anchors the nail in place when fluid is injected into said hollow region.

28. Apparatus according to claim 27 wherein the channel is linked to inject fluid into the hollow region.

29. Apparatus according to claim 27 wherein the channel is linked to inject cement to bond in place one or both of the nail and the first screw.

30. Apparatus according to claim 1, and including a motion limiting element to prevent the locking mechanism from separating from the nail.

31. Intramedullary nail apparatus comprising:
an intramedullary nail with an axis, a proximal end, a distal end, and a first hole going through said nail at an angle to the axis;
a first screw which goes through the first hole;
a locking mechanism, operative to lock in place the first screw; and
a motion limiting element that prevents the locking mechanism from separating from the nail.

32. Apparatus according to claim 30, wherein at least part of the locking mechanism is inside the nail, and the motion limiting element comprises:
a nail-stopping screw;
a nail-stopping screw hole in the side of the nail going from the outside of the nail to the inside of the nail; and
a nail-stopping indentation in the side of the locking mechanism.
wherein the nail-stopping screw goes through the nail-stopping screw hole and engages the nail-stopping indentation when the motion limiting element operates to prevent the locking mechanism from separating from the nail.

33. Apparatus according to claim 32 wherein the nail-stopping indentation is longer in the direction of the nail axis than the width of the portion of the nail-stopping screw that engages in said slot, and the motion limiting element has at least one mode of operation in which the locking mechanism is free to move axially by a given distance but is prevented from separating from the nail.

34. Apparatus according to claim 32 wherein the nail-stopping indentation comprises a slot of limited width in the azimuthal direction.

35. Apparatus according to claim 32 wherein the nail-stopping indentation extends around the entire circumference of the locking mechanism.

36. Apparatus according to claim 1, wherein locking the first screw comprises not allowing the first screw to move in the direction of its axis.

37. Apparatus according to claim 1, wherein locking the first screw comprises allowing the first screw to move only a limited distance in the direction of its axis.

38. A method for installing an intramedullary nail in the proximal femur, comprising:
inserting the nail, distal end first, into the medullar channel of the femur from the head of the femur;
inserting a first screw through a first hole in the nail, through the head of the femur and through the trochanter, at an angle to the axis of the nail;
inserting a second screw through a second hole in the nail, closer to the proximal end of the nail than the first screw; and
locking the first screw in place using a locking mechanism accessible from the proximal end of the nail.
